(19) United States
(12) Patent Application Publication
(10) Pub. No.: US 2026/0295133 A1
(43) Pub. Date: Oct. 1, 2026

(54) CONNECTION PIECE AND SUBSTANCE EXCHANGE DEVICE WITH CONNECTION PIECE

(71) Applicant: enmodes GMBH, Aachen (DE)

(72) Inventors: Ilse Philine RITTER, Aachen (DE); Ralf BORCHARDT, Aachen (DE)

(21) Appl. No.: 19/489,875

(22) PCT Filed: Jul. 26, 2024
(86) PCT No.: PCT/EP2024/071254

§ 371 Date: Dec. 3, 2025

(30) Foreign Application Priority Data
Aug. 30, 2023 (DE) 10 2023 123 307.6

(51) Int. Cl. A61M 1/14; A61M 1/16; A61M 39/10; F16K 5/08

(52) U.S. Cl. CPC A61M 1/152; A61M 1/155; A61M 1/1698; A61M 39/1011; F16K 5/08

(57) ABSTRACT

A connecting piece for connecting a blood pump to a mass transfer unit comprises an elastic hose element having an inflow connection, which in particular can be attached to the pressure connector of a blood pump, and a discharge connection, which in particular can be attached to the blood inlet port of a mass transfer unit, wherein, proceeding from the inflow connection, the hose element comprises a hose section ascending in height, which transitions via an elbow into a hose section descending in height, the end of which is formed by the discharge connection, and wherein a removal channel is disposed in the elbow, in particular at the highest point thereof.

The invention relates to a connecting piece for connecting a blood pump to a mass transfer unit comprising an elastic hose element having an inflow connection, which in particular can be attached to the pressure connector of a blood pump, and having a discharge connection, which in particular can be attached to the blood inlet port of a mass transfer unit.

The invention also relates to a mass transfer device for blood, comprising a blood pump for pumping blood and a mass transfer unit for treating the blood pumped by the blood pump, in particular for treatment by exchanging a substance between the blood and a second transfer medium.

In the invention, such a mass transfer unit can preferably be formed by a gas exchanger, in particular in which blood can be enriched with oxygen and from which carbon dioxide can be eliminated.

A mass transfer unit of the invention has, for example, a chamber that is divided into two chamber regions by at least one mass-permeable, in particular gas-permeable, membrane, wherein blood can be passed through one of the chamber regions and a second transfer medium, preferably a gas, can be passed through the other, with which a mass transfer, preferably a gas exchange, is to take place through the at least one membrane. Each chamber region preferably has inlet and outlet ports provided for the particular medium (blood/second transfer medium or gas). According to the invention, the blood inlet port of the mass transfer unit can preferably be connected to the discharge connection of the connecting piece.

Connecting pieces of the described type are often designed as simple pieces of hose in the prior art. This can lead to problems in that venting of the hose and of the connected device parts is not always reliably ensured. Furthermore, the removal of blood samples from a device connected to the hose is problematic. For this purpose, a rigid PC connector having a Luer connection is often installed in the prior art, which promotes thrombus growth, both at the Luer connection itself and at the transition between the hose and the PC connector.

Another disadvantage is that simple hoses are often used in prefabricated lengths that are too long for the application, which adversely affects organization and unnecessarily increases the blood volume, thereby compromising the hemocompatibility of the entire system.

Against this background, it is an object of the invention to provide a connecting piece which overcomes these disadvantages, in particular which offers reliable venting of the blood being transported and, preferably, also an option for taking samples.

A further object is to improve a mass transfer device of the type mentioned at the outset comprising a connecting piece according to the invention.

This object is achieved by a connecting piece of the type mentioned at the outset, in which, proceeding from the inflow connection, the hose element comprises a hose section ascending in height, which transitions via an elbow into a hose section descending in height, the end of which is formed by the discharge connection, with a removal channel being disposed in the elbow.

In a preferred embodiment, such a removal channel can be disposed, for example, at the highest point of the elbow and/or can at least open into the elbow at the highest point.

In another preferred embodiment, it may also be provided that the removal channel is offset laterally from the highest point of the elbow, in particular so as to be disposed below the highest point of the elbow and/or at least open into the elbow there. This can, for example, promote ergonomic blood removal through the removal channel. It may then be provided that, in order to remove gas bubbles from the elbow, the connecting piece is tilted so as to thereby, solely for the purpose of removing the gas bubbles, move the removal channel and/or at least the opening thereof into the elbow to the highest point of the elbow in the tilted state.

Such a connecting piece is preferably inherently stable, which is to say, despite preferably having flexibility/elasticity, having a predetermined shape or a predetermined progression of the type described when no external forces act on the connecting piece. The connecting piece is preferably made of at least one, in particular hemocompatible, flexible plastic material, for example, silicone or PVC, or several silicone components.

The aforementioned progressions are present at least during an intended use of the connecting piece when the same is disposed between a blood pump and a mass transfer unit, and these elements are disposed in the space in an operational position.

The invention ensures that, during the intended use, any bubble-shaped gas components in the blood collect in the elbow of the connecting piece, which forms the highest point of the connecting piece. Since the hose sections on both sides of the elbow descend in height proceeding from the elbow, any gas bubbles also remain in the elbow when the blood flow stops or slows down. According to the invention, the removal channel in the elbow thus provides the option of eliminating a gas bubble from the connecting piece, in particular by simply opening the removal channel or by suction through the removal channel. The removal channel can likewise be used to draw blood samples from the connecting piece through the removal channel. The removal channel can thus fulfill several functions.

The inherent stability that is preferably present furthermore ensures that a blood pump and a mass transfer unit connected thereto maintain a position with respect to one another which is defined by the inherent stability of the connecting piece.

In a preferred embodiment, the invention provides for a valve to be attached to the removal channel or for a valve to be integrated into the removal channel. Such a valve makes it very easy to open and close the removal channel again so as to fulfill a desired function, for example, venting the connecting piece or taking a blood sample.

When the valve is integrated into the removal channel, it is preferable that the removal channel forms at least part of, and in particular the entirety of, the housing of the valve, in which a valve element of the valve is rotatably mounted. The removal channel can, for example, include a region having an enlarged cross-section in which the valve element rotatably rests.

Such a valve element can, for example, be an element having a circular cross-section, in particular a pin element, which has a radially extending through-borehole that can be aligned with or oriented transversely to the removal channel by rotation.

It is preferably provided that the valve element is regionally disposed in the upper wall region of the elbow. Based on the diameter of the rotatable valve element, it may preferably be provided that the valve is disposed over at least 10% of the diameter thereof in the upper wall region of the elbow.

Such an at least partial integration of the valve element into the upper wall of the elbow also achieves a high degree of stability for the valve thus implemented. The upper wall region of the elbow and the removal channel then preferably in part form a housing of the valve element.

In all possible specific embodiments, it may preferably be provided that the removal channel comprises an attachment element, preferably a Luer lock, at the free end. This allows standardized medical components to be attached to the removal channel.

Furthermore, it is preferable in all possible embodiments for the inflow connection and/or the discharge connection on the inner side of the hose to have a form-locking structure for attaching a hose nozzle. This ensures a secure connection to the blood pump and/or to the gas exchanger if these have correspondingly designed hose nozzles.

According to a preferred embodiment, the length of the hose section ascending in height between the inflow connection and the elbow is smaller than the length of the hose section descending in height between the elbow and the discharge connection. This allows for, for example, a preferred embodiment in which a blood pump is disposed in the upper region or even completely above a mass transfer unit in the case of a mass transfer device that is implemented with the connecting piece. This results in particularly good organization of all components.

However, other configurations in which the length ratios are reversed or both hose sections on either side of the elbow have the same length may also be present.

According to a preferred embodiment of the invention, the removal channel is, and in particular, regions of the elbow immediately adjoining the removal channel also are, made of the same, preferably elastomeric material as the remaining region of the elbow and the adjoining hose sections. In this case, the connecting piece can be produced especially easily from a single material, for example, by injection molding.

According to an alternative embodiment, the removal channel is, in particular also regions of the elbow immediately adjoining the removal channel are, made of a different, preferably elastomeric material than the remaining region of the elbow and the adjoining hose sections. For example, a firmer and/or less elastic material can be selected for the removal channel and possibly adjoining regions of the elbow compared to the remaining regions of the elbow and the adjoining hose sections. Manufacturing can be carried out, for example, by 2-component injection molding. This ensures that the regions that are subject to greater stress during use are more stable.

Preferably, the removal channel and the elbow, as well as the adjoining hose sections, are designed in one piece, in particular manufactured using 2-component injection molding, and preferably are integrally joined to one another.

However, an embodiment may also be provided in which the removal channel is designed separately from the elbow (and the adjoining hose sections) and is detachably fastened to the elbow.

In such an embodiment, an outwardly protruding receiving region is preferably integrally molded on the elbow, in particular an undercut receiving region is integrally molded on.

A rotatable valve element, for example, is inserted, or at least can be inserted, into this receiving region, wherein the removal channel, in particular at least the free end region thereof, comprises or forms a bell which covers the valve element and the receiving region and which is fastened, or at least can be fastened, to the receiving region. Such fastening can be carried out, for example, by latching engagement of the bell on the receiving region.

Alternatively, the removal channel, and in particular the removal channel comprising a valve, is inserted, or at least can be inserted, into the receiving region, and preferably can be fastened therein so as to be clamped, with a channel section facing the elbow. For secure fastening, a securing element, for example a cable tie, can be placed around the receiving region so as to exert a radial securing force.

If the removal channel is designed separately from the elbow (and the adjoining hose sections), it may also be preferable, in another embodiment, to provide a fastening opening on the elbow to or in which the removal channel is fastened, or at least can be fastened. The fastening opening is preferably designed as a through-opening which opens into the lumen of the elbow, in particular with the fastening openings being disposed in a wall region of the elbow that is at least not thickened compared to other adjoining elbow regions.

Furthermore, the removal channel comprises a collar at the end region facing the elbow, wherein in a possible embodiment the collar is inserted, or at least can be inserted, through the opening into the interior of the elbow, wherein the collar can latch over the edge of the opening and a detachable counter element that is located opposite the collar is provided, and wherein an edge region surrounding the opening is enclosed, or at least can be enclosed, between the collar and the counter element. On the side of the openings facing the lumen of the elbow, the wall thickness of the elbow can be reduced in an annular region surrounding the openings, thereby forming a recess in which the collar is accommodated, or at least can be accommodated, in particular without protruding beyond the inner surface of the elbow. For example, the collar can be concave on the side facing the lumen of the elbow, preferably with the curvature of the collar being adapted to the progression of the curvature of the inner surface of the elbow.

Alternatively, it is preferably provided that the collar is fastened in the thickness of an upper wall region of the elbow, is preferably embedded therein in an integral and/or form-locked manner, preferably with a radially inwardly open annular groove being provided around the opening in the upper wall region, into which the collar is inserted.

The annular groove is preferably disposed within the thickness of the wall region of the elbow so that material of the wall region is present beneath and above the annular groove.

Using a connecting piece according to the invention, a mass transfer device for blood can preferably be formed, which comprises a blood pump for pumping blood and a mass transfer unit for treating the blood pumped by the blood pump, wherein the pressure connector of the blood pump is connected to the blood inlet port of the mass transfer unit via the connecting piece.

The blood pump and the mass transfer unit can preferably be fastened to one another, in particular in a stable position. Fastening may also be carried out solely by way of the connecting piece or by additional connecting elements.

Furthermore, it is preferably provided that the blood pump is fastened to the upper housing cover of the mass transfer unit, and in particular next to the blood inlet port thereof, preferably with the blood pump being integrated into the wall of the mass transfer unit cover. The blood pump can likewise be fastened to the lateral housing wall of the mass transfer unit extending between the bottom and top sides of the mass transfer unit, preferably with the blood pump being integrated into the housing wall.

Specific embodiments of the invention will be described in more detail based on the figures.

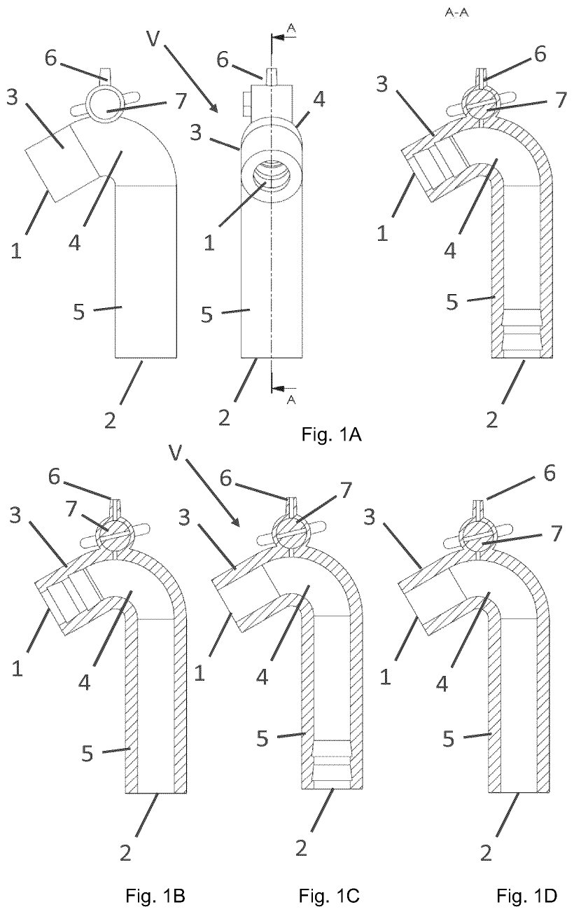

FIGS. 1 to 7 show different variants of a first embodiment of a connecting piece V according to the invention.

These variants have the features described below in common, unless the differences are expressly pointed out.

In all instances, the connecting piece V according to the invention is formed by a hose element, which has an inflow connection 1 at one end and a discharge connection 2 at the other end. In the arrangement of the connecting piece V shown here, in each case in the intended use, the hose element, proceeding from the inflow connection 1, comprises a hose section 3 that ascends in height and transitions via an elbow 4 into a hose section 5 that descends in height, the end of which is formed by the discharge connection 2, wherein a removal channel 6 is disposed in the elbow 4, preferably at the highest point thereof. This removal channel is used to remove blood from the connecting piece and is likewise suitable for removing or releasing air bubbles that may collect in the elbow 4 when blood flows through the connecting piece V.

In all the embodiments shown in FIGS. 1 to 7, the ascending hose section 3 is shorter than the descending hose section 5. This is only an exemplary illustration. The length ratios can also be reversed, or the lengths can be the same.

The details in which the connecting pieces V of the different embodiments differ are described below.

FIGS. 1A to 1D show variants of a specific embodiment in which a valve is integrated into the removal channel 6, wherein the valve comprises a rotatable valve element 7 disposed regionally in the upper wall region of the elbow 4. For this purpose, the removal channel 6 has an enlarged cross-section in which the valve element 7 rotatably rests. The valve element has a circular cross-section (in all embodiments). Based on the diameter of the valve, it may be provided that the valve element 7 is integrated over at least 10% of the diameter thereof into the thickness of the wall region.

In the variant shown in FIG. 1A, both the inflow connection 1 and the discharge connection 2 on the inside of the hose have a form-locking structure for attaching a hose nozzle. In FIG. 1B, only the inflow connection 1 has such a form-locking structure for attaching a hose nozzle, in FIG. 1C only the discharge connection 2 has such a structure, and in FIG. 1C neither of the connections has such a structure.

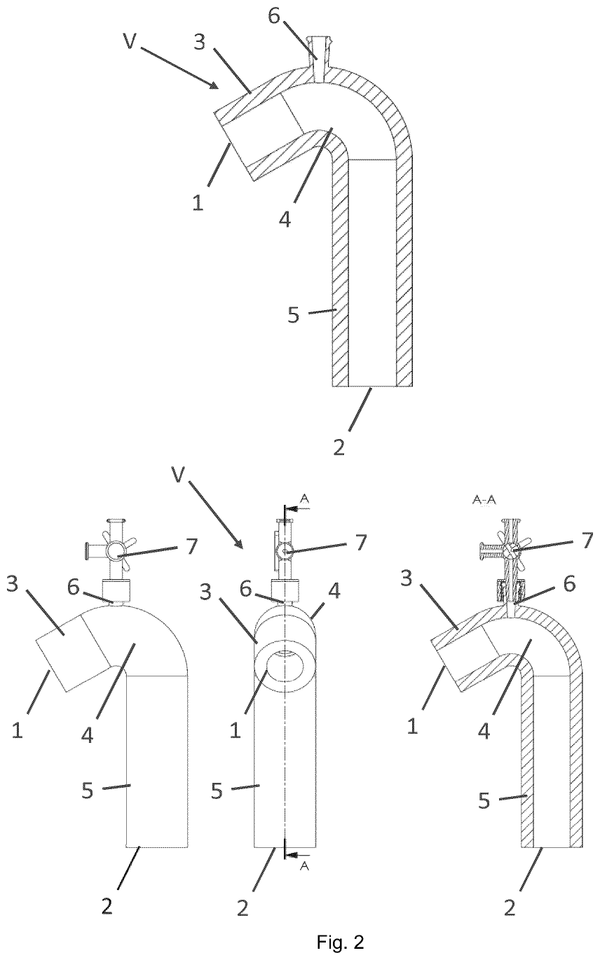

This type of design having form-locking structures can also be provided in all other specific embodiments of FIGS. 2 to 7, even though the connections 1 and 2 are shown there without form-locking structures.

In FIG. 2, the embodiment is such that the removal channel 6 itself does not comprise a valve, but one can be fastened thereto. For example, the removal channel 6 comprises an attachment element, preferably a Luer lock, at the free end thereof for this purpose. A fluid line element including a valve 7 can be attached/screwed to this attachment element.

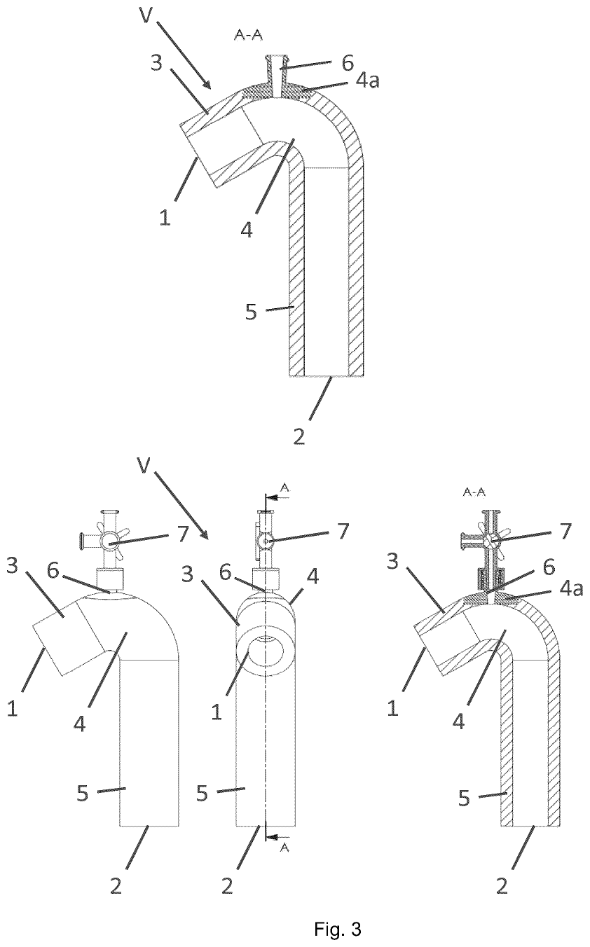

The specific embodiment of FIG. 3 differs from that of FIG. 2 in that the removal channel 6 is, and in particular regions 4a of the elbow 4 immediately adjoining the removal channel also are, made of a different, preferably firmer and/or less elastic material than the remaining region of the elbow 4 and the adjoining hose sections 3 and 5, preferably an elastomeric material.

In FIGS. 1 and 2, in contrast, the removal channel 6 is, and in particular regions 4a of the elbow 4 immediately adjoining the removal channel also are, made of the same, preferably elastomeric material as the remaining region of the elbow 4 and the adjoining hose sections 3, 5.

In all instances, silicone or PVC can preferably be selected as the material, and in particular materials having differing hardness levels can be selected, if the connecting piece V is not made of one material, as in FIG. 3.

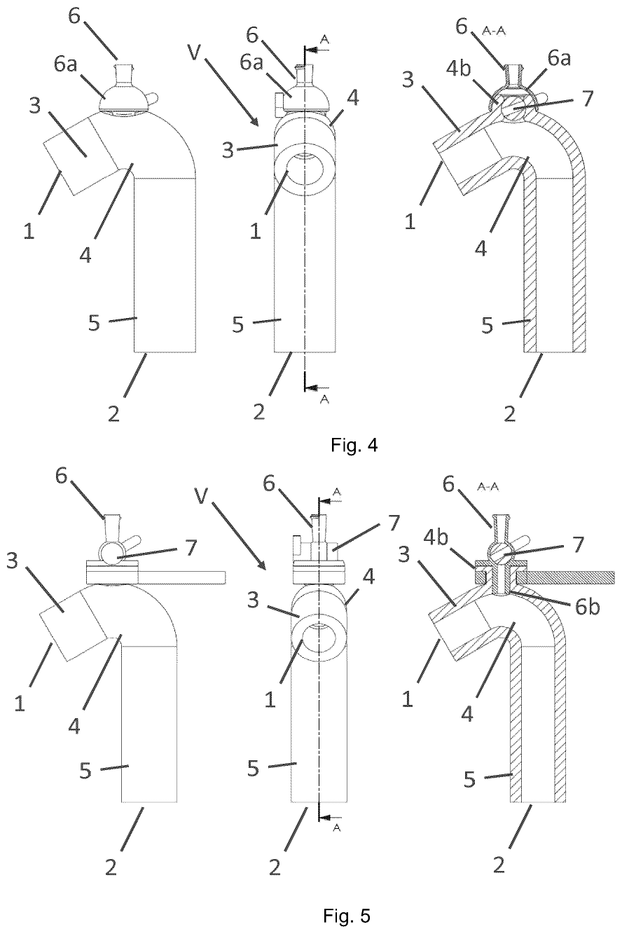

According to the embodiments of FIGS. 4 to 7, in various variants the removal channel 6 is designed separately from the elbow 4; the removal channel 6 and the elbow 4 are thus, at least initially, separately manufactured components. In FIGS. 4 to 6, the removal channel 6 can be detachably fastened to the elbow 4, for example, while in FIG. 7, it can also be integrally joined to the elbow, for example by adhesive bonding.

In FIGS. 4 and 5, an outwardly protruding receiving region 4b is integrally molded on the elbow 4, in particular an undercut receiving region 4b.

FIG. 4 shows an embodiment in which a rotatable valve element 7 is inserted into the receiving region 4b, and the removal channel 6 comprises or forms a bell 6a which covers the valve element 7 and the receiving region 4b, and which is fastened to the receiving region 4b.

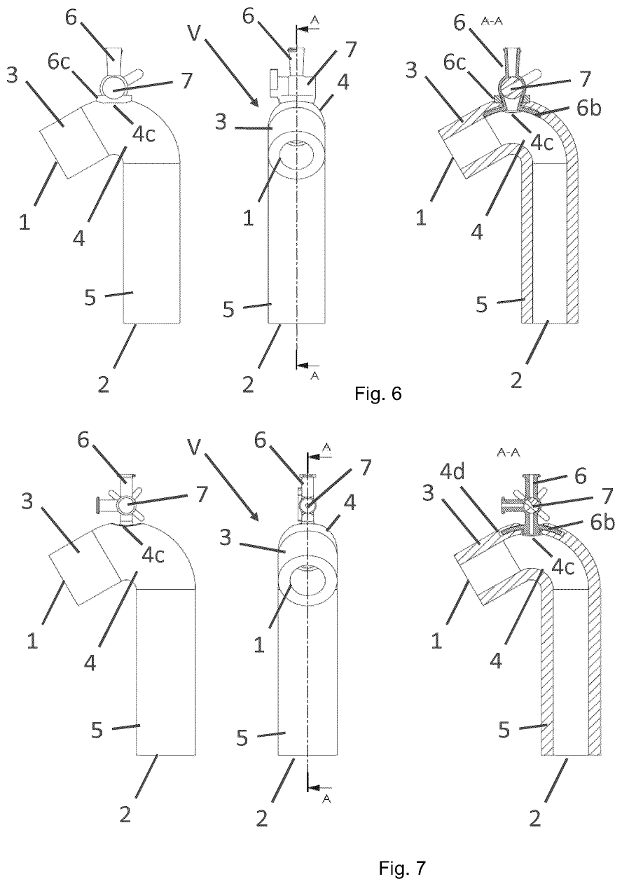

In FIG. 5, the removal channel 6 comprising a valve is inserted into the receiving region 4b, which is an undercut receiving region here, with a channel section 6b facing the elbow, and preferably is fastened therein so as to be clamped. The fastening is carried out, for example, by a cable tie. In FIGS. 6 and 7, a fastening opening 4c is provided at the elbow 4 to which, or in which, the removal channel 6 is fastened, wherein the removal channel 6 comprises a collar 6b at the end region thereof facing the elbow 4.

According to the embodiment shown in FIG. 6, the collar 6b is inserted through the opening 4c into the interior of the elbow 4, which is to say the lumen thereof, wherein the collar 6b latches over the edge of the opening 4c. Furthermore, a preferably detachable counter element 6c, which is located opposite the collar 6b, is provided, wherein an edge region surrounding the opening 4c is enclosed between the collar 6b and the counter element 6c.

In the embodiment of FIG. 7, the collar 6b is fastened in the thickness of an upper wall region of the elbow 4, and preferably is embedded therein in an integral and/or form-locked manner, with a radially inwardly open annular groove 4d being provided around the opening 4c in the upper wall region, into which the collar 6b is inserted. For an integral bond, this annular groove 4d can be filled with an adhesive before the removal channel 6 is inserted.

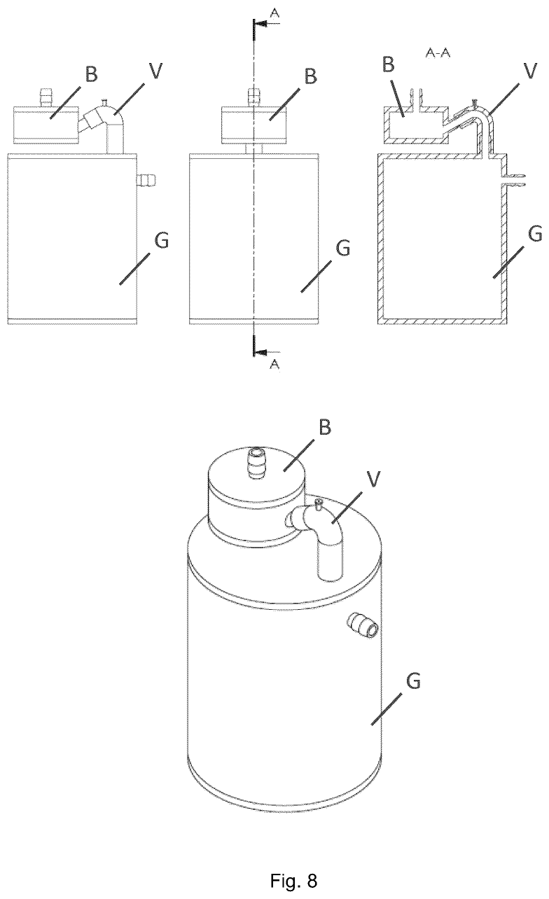

FIGS. 8 to 11 show various uses of the connecting piece V, each in a mass transfer device for blood comprising a blood pump B for pumping blood and a mass transfer unit G for treating the blood pumped by the blood pump B by way of mass transfer with a second transfer medium, wherein the pressure connector of the blood pump B is connected to the blood inlet port of the mass transfer unit G via a connecting piece V. In particular, further blood transport is not shown, and in particular the lines that are run and the ports that are used for the second transfer medium, in particular a gas, are also not shown.

In the embodiment shown in FIG. 8, the blood pump B is fastened above the upper housing cover of the mass transfer unit G, in particular is fastened thereto, here next to the blood inlet port thereof.

It may also be provided that, although not shown here, the blood pump B is integrated into the wall of the mass transfer unit cover. For example, the blood pump B may only be fastened to the cover of the mass transfer unit G via the connecting piece or additionally via connecting elements, for example, which extend between the lower surface of the blood pump B and the upper surface of the housing cover of the mass transfer unit G.

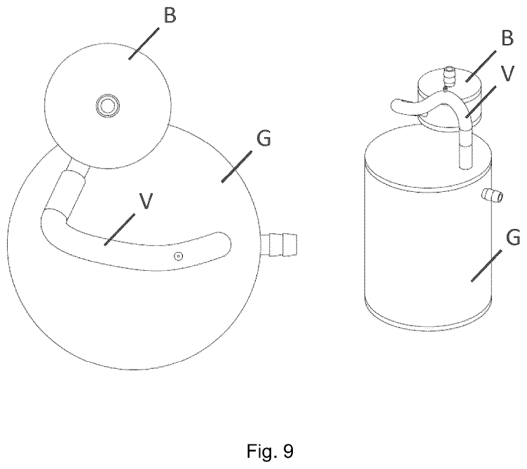

In FIG. 9, the blood pump B is also located above the mass transfer unit G, but only partially thereabove. Here too, the blood pump B may, for example, only be fastened to the cover of the mass transfer unit G via the connecting piece or additionally via connecting elements, for example, which extend between the lower surface of the blood pump B and the upper surface of the housing cover of the mass transfer unit G.

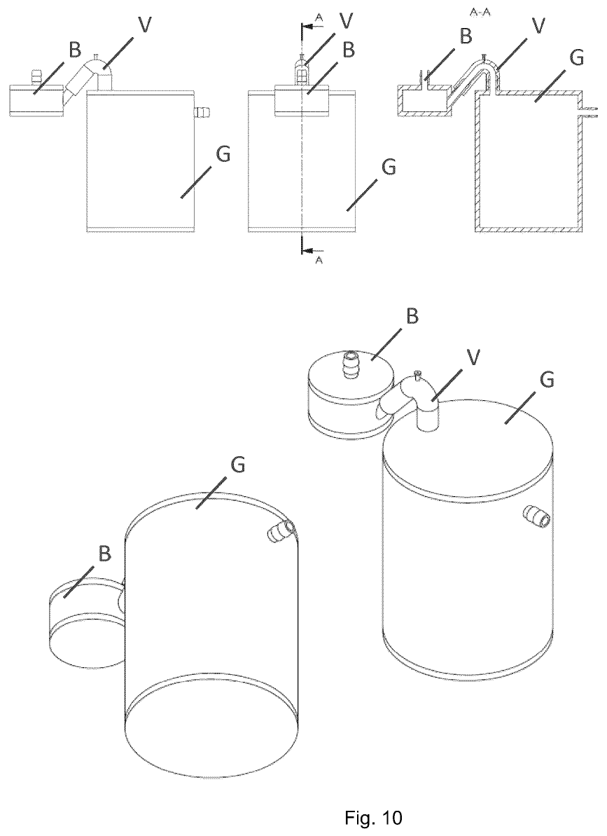

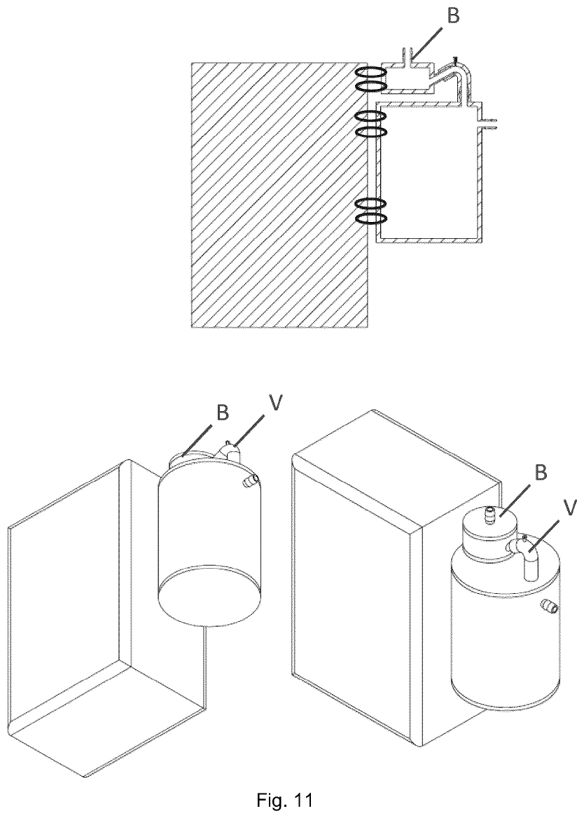

FIG. 10 shows an embodiment in which the blood pump B is disposed laterally next to the mass transfer unit. Here, the blood pump B can be fastened to the lateral housing wall of the mass transfer unit G between the bottom and top sides of the mass transfer unit, for example by way of connecting elements that extend between these regions.

FIG. 11 shows an embodiment in which the mass transfer device comprising a blood pump B, a connecting piece V, and a mass transfer unit G is fastened to a further element, for example, a console. For example, the blood pump B and mass transfer unit G alone may have connections to the further element.

All embodiments show that a connecting piece according to the invention can be used to create a user-friendly arrangement of the blood pump to the mass transfer unit, wherein the connecting piece combines the functions of venting and blood removal.

1. A connecting piece for connecting a blood pump to a mass transfer unit comprising an elastic hose element having an inflow connection configured to be attached to pressure connector of a blood pump and a discharge connection configured to be attached to a blood inlet port of a mass transfer unit wherein, proceeding from the inflow connection the hose element comprises a first hose section ascending in height, which transitions via an elbow into a second hose section descending in height, the end of which is formed by the discharge connection a removal channel being disposed in the elbow at a highest point thereof.

2. The connecting piece according to claim 1, wherein a valve is attached to or integrated into the removal channel, the valve comprising a rotatable valve element which is disposed in an upper wall region of a wall of the elbow, at least 10% of a diameter of the rotable valve element being within the upper region of the wall of the elbow.

3. The connecting piece according to claim 1, wherein the removal channel comprises an attachment element, comprising a Luer lock, at a free end thereof.

4. The connecting piece according to claim 1, thatwherein the inflow connection and/or the discharge connection on an inner side of the first and/or second respective hose section have a form-locking structure for attaching a hose nozzle.

5. The connecting piece according to claim 1, wherein a length of the first hose section is shorter than a length of the second hose section.

6. The connecting piece according to claim 1, wherein walls of the removal channel and regions of the elbow immediately adjoining the removal channel are
a. made of a same, elastomeric material as remaining regions of the elbow and the first and second hose sections, or
b. made of a firmer and/or less elastic elastomeric material than the remaining regions of the elbow and the first and second hose sections.

7. The connecting piece according to claim 1, wherein the removal channel and the elbow as well as the first and second hose sections are formed in one piece by 2-component injection molding.

8. The connecting piece according to claim 1, wherein the removal channel and the elbow are separate pieces, the removal channel being detachably fastened to the elbow.

9. The connecting piece according to claim 8, wherein an undercut, outwardly protruding receiving region is integrally molded on the elbow,
a. in which receiving region a rotatable valve element is inserted, and the removal channel comprises a bell-shaped element which covers the valve element and the receiving region and which is fastened, to the receiving region, or
b. in which receiving region a valve comprising the removal channel is inserted, so as to be clamped, with a section of removal channel facing the elbow.

10. The connecting piece according to claim 8, wherein a fastening opening is provided at the elbow, to which, or in which, the removal channel is fastened, the removal channel further comprising a collar an end region thereof facing the elbow, and
a. the collar is inserted, through the fastening opening into an interior of the elbow, the collar being latchable over an edge of the opening, and a detachable counter element that is located opposite the collar being provided, an edge region surrounding the opening being enclosed between the collar and the counter element, or
b. the collar embedded in an upper wall region of the elbow in an integral and/or form-locked manner, with a radially inwardly open annular groove being provided around the opening in the upper wall region of the elbow, into which the collar is inserted.

11. A mass transfer device for blood, comprising a blood pump for pumping blood and a mass transfer unit for treating the blood pumped by the blood pump wherein a pressure connector of the blood pump is connected to a blood inlet port of the mass transfer unit via a connecting piece according to claim 1.

12. The mass transfer device according to claim 11, wherein the blood pump and the mass transfer unit are stably fastened to one another.

13. The mass transfer device according to claim 12, wherein the blood pump is integrated into a wall of upper, housing cover of the mass transfer unit, next to the blood inlet port thereof.

14. The mass transfer device according to claim 12, wherein the blood pump is integrated into a lateral housing wall of the mass transfer unit extending between bottom and top sides of the mass transfer unit.
